(19) United States
(12) Patent Application Publication
(10) Pub. No.: US 2021/0103988 A1
(43) Pub. Date: Apr. 8, 2021

(54) SYSTEMS AND METHODS FOR PROCESSING DATABASE QUERIES

(71) Applicant: The Toronto-Dominion Bank, Toronto (CA)

(72) Inventors: Stuart MARSHALL, Milton (CA); Mark Andrew HARDY, Stoney Creek (CA)

(73) Assignee: The Toronto-Dominion Bank, Toronto (CA)

(21) Appl. No.: 16/592,838

(22) Filed: Oct. 4, 2019

(51) Int. Cl. G06Q 40/06; G16H 10/60; G16H 20/10; G06K 9/34; G06F 16/953

(52) U.S. Cl. CPC G06Q 40/06; G16H 10/60; G06K 2209/01; G06K 9/344; G06F 16/953; G16H 20/10

(57) ABSTRACT

A method for processing results of database queries is disclosed. The method includes: receiving, from a client device, a request to obtain a rating for a user based on medical information associated with the user; receiving, via a user interface on the client device, input of identifying information for medication associated with the user; generating a database query for retrieving a first data set associated with the identified medication, the first data set containing one or more data parameters that are determined based on a type of the requested rating; transmitting the database query to a database storing data associated with a plurality of different medications; receiving, from the database, results of the database query, the results including multiple possible values for at least one of the data parameters; generating a medication profile for the user based on the received results, the medication profile defining probabilities associated with possible values for the at least one data parameter of the results; and obtaining the requested rating based on the medication profile for the user.

### TECHNICAL FIELD

The present disclosure relates to data processing, and more particularly, to systems and methods for processing database queries.

### BACKGROUND

Computing systems associated with insurance product providers typically require prospective customers to input health conditions. The inputted information may be difficult to parse for health condition information since customers may not know precise medical names of conditions that affect them. Further, there may be wide variation in the description of health conditions that is inputted by different users, making it difficult to have a unified approach for processing user-inputted data.

It would be desirable to provide a system that supports consistent capture of user's medical information and subsequent downstream processing of the medical information, in a manner that can reduce the likelihood of error in identifying health conditions and consumption of processing resources.

### BRIEF DESCRIPTION OF THE DRAWINGS

Reference will now be made, by way of example, to the accompanying drawings which show example embodiments of the present application and in which:

FIG. 1 is a schematic operation diagram illustrating an operating environment of an example embodiment;

FIG. 2 is a high-level schematic diagram of an example computing device;

FIG. 3 shows a simplified organization of software components stored in memory of the example computing device of FIG. 2;

FIG. 4 shows, in flowchart form, an example method for processing database queries associated with a user's medication;

FIG. 5 shows, in flowchart form, another example method for processing database queries associated with a user's medication; and

FIG. 6 shows, in flowchart form, another example method for processing database queries associated with a user's medication.

Like reference numerals are used in the drawings to denote like elements and features.

### DETAILED DESCRIPTION OF EXAMPLE EMBODIMENTS

In an aspect, the present disclosure describes a computing system. The computing system includes a communications module communicable with an external network, a memory, and a processor coupled to the communications module and the memory. The processor is configured to: receive, from a client device, a request to obtain a rating for a user based on medical information associated with the user; receive, via a user interface on the client device, input of identifying information for medication associated with the user; generate a database query for retrieving a first data set associated with the identified medication, the first data set containing one or more data parameters that are determined based on a type of the requested rating; transmit the database query to a database storing data associated with a plurality of different medications; receive, from the database, results of the database query, the results including multiple possible values for at least one of the data parameters; generate a medication profile for the user based on the received results, the medication profile defining probabilities associated with possible values for the at least one data parameter of the results; obtain the requested rating based on the medication profile for the user.

In some implementations, the one or more data parameters may include a medical condition parameter identifying possible medical conditions associated with the identified medication.

In some implementations, the processor may be further configured to, after retrieving possible values for the medical condition parameter: provide, to the client device, the possible medical conditions associated with the identified medication; and receive, from the client device, input of user selection of a first medical condition from among the possible medical conditions.

In some implementations, the medication profile may include, for each of at least one of the possible medical conditions, a prevalence score indicating a prevalence of the medical condition.

In some implementations, the one or more data parameters may include a mortality rate parameter identifying possible mortality rates associated with the identified medication.

In some implementations, obtaining the requested rating may comprise determining that the user is eligible for insurance coverage.

In some implementations, obtaining the requested rating may comprise determining an insurance premium for the user.

In some implementations, the processor may be further configured to transmit, to a server, a request to obtain medication information for the user.

In some implementations, the user interface may allow input of image data containing identifying information for the medication associated with the user.

In some implementations, the processor may be further configured to perform optical character recognition based on user inputted image data to obtain a unique identifier associated with the medication.

In another aspect, the present disclosure describes a processor-implemented method for processing results of database queries. The method includes: receiving, from a client device, a request to obtain a rating for a user based on medical information associated with the user; receiving, via a user interface on the client device, input of identifying information for medication associated with the user; generating a database query for retrieving a first data set associated with the identified medication, the first data set containing one or more data parameters that are determined based on a type of the requested rating; transmitting the database query to a database storing data associated with a plurality of different medications; receiving, from the database, results of the database query, the results including multiple possible values for at least one of the data parameters; generating a medication profile for the user based on the received results, the medication profile defining probabilities associated with possible values for the at least one data parameter of the results; and obtaining the requested rating based on the medication profile for the user.

Other example embodiments of the present disclosure will be apparent to those of ordinary skill in the art from a review of the following detailed descriptions in conjunction with the drawings.

In the present application, the term “and/or” is intended to cover all possible combinations and sub-combinations of the listed elements, including any one of the listed elements alone, any sub-combination, or all of the elements, and without necessarily excluding additional elements.

In the present application, the phrase “at least one of . . . or . . . ” is intended to cover any one or more of the listed elements, including any one of the listed elements alone, any sub-combination, or all of the elements, without necessarily excluding any additional elements, and without necessarily requiring all of the elements.

For an ordinary individual, accurately identifying health conditions that they are experiencing may be challenging. The individual may not be aware of the scientific or medical name of the specific health conditions. Descriptions of health conditions may vary widely from person-to-person, making it difficult for the processing system to identify health conditions based on the descriptions. For example, the language used to describe the same health condition may differ significantly among different individuals.

The present disclosure describes improved methods for capturing medical history data of users of a computing system. A computing system receives, from a client device, a request to obtain a rating for a user of the client device based on their medical information. A user interface is provided on the client device to facilitate input of identifying information for medication associated with the user. A database query is generated for retrieving a data set associated with the identified medication, where the data set includes one or more data parameters that are determined based on a type of the requested rating. The query is transmitted to a database which stores data associated with a plurality of different medications, and the computing system receives the results of the query. The results include multiple possible values for at least one of the data parameters included in the retrieved data set. Based on the received query results, the computing system generates a medication profile for the user. The medication profile defines probabilities associated with possible values for the at least one data parameter of the query results. The requested rating is then obtained based on the medication profile for the user.

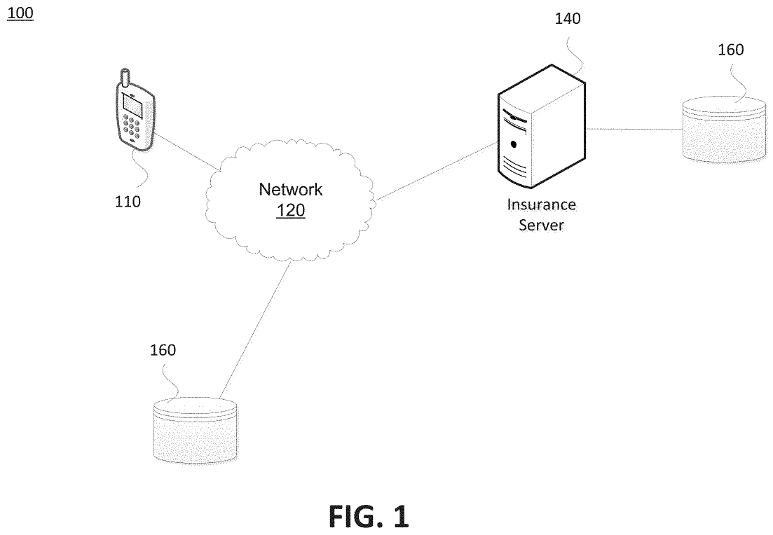

FIG. 1 is a schematic diagram of an exemplary operating environment in accordance with embodiments of the present disclosure. FIG. 1 illustrates a system 100 for processing database queries relating to medications for a specific user.

As illustrated, an insurance server 140 and client device 110 communicate via the network 120. The client device 110 is a computing device that may be associated with an entity, such as a user or client. The insurance server 140 is coupled to a database 150, which may be provided in secure storage. The secure storage may be provided internally within the insurance server 140 or externally. The secure storage may, for example, be provided remotely from the insurance server 140.

The database 150 stores secure data. In particular, the database 150 may include records for a plurality of insurance policy accounts associated with customer entities. That is, the secure data may comprise insurance policy account data for one or more customer entities. The secure data may include personal data, such as personal identification information. The personal identification information may include any stored personal details associated with a customer entity including, for example, name, age, date of birth, gender, a home, work or mailing address, contact information such as a messaging address (e.g. email address), and/or a telephone number, a government-issued identifier such as a social insurance number (SIN) and/or driver's license number, etc.

In some embodiments, the database 150 may be a computer system that includes one or more database servers, computer servers, and the like. In some embodiments, the database 150 may be an application programming interface (API) for a web-based system, operating system, database system, computer hardware, or software library.

The insurance server 140 may be associated with an insurance company (and, more generally, a merchant of insurance policies). The insurance server 140 may maintain data associated with insurance policies that are provided by the insurance company. For example, the insurance server 140 may store or be connected to a database, such as database 150, containing policy data for a plurality of insurance policies.

The system 100 also includes a medications database 160. The medications database 160 includes data associated with various different medications. The medications database 160 may contain identifiers for a plurality of medications and information corresponding to those medications. For example, the medications database 160 may include medical data such as, without limitation, health conditions which may be treated using the medications, recommended or preferred dosages of the medications for various health conditions, demographics of users of the medications, mortality rates for users of the medications, and channels of sale or prescription.

The client device 110, the insurance server 140, and the medications database 160 may be in geographically disparate locations. Put differently, the client device 110 may be remote from one or both of the insurance server 140 and the medications database 160.

The client device 110 and the insurance server 140 are computer systems. The client device 110 may take a variety of forms including, for example, a mobile communication device such as a smartphone, a tablet computer, a wearable computer such as a head-mounted display or smartwatch, a laptop or desktop computer, or a computing device of another type.

The network 120 is a computer network. In some embodiments, the network 120 may be an internetwork such as may be formed of one or more interconnected computer networks. For example, the network 120 may be or may include an Ethernet network, an asynchronous transfer mode (ATM) network, a wireless network, or the like.

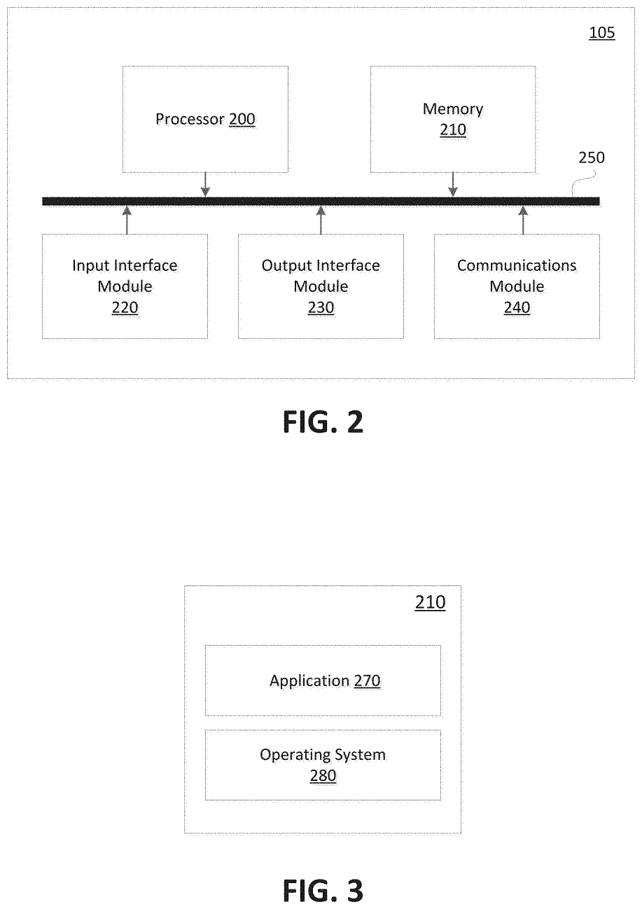

FIG. 2 is a high-level operation diagram of the example computing device 105. In some embodiments, the example computing device 105 may be exemplary of one or more of the client device 110, the insurance server 140, and the third-party application server 180. The example computing device 105 includes a variety of modules. For example, as illustrated, the example computing device 105, may include a processor 200, a memory 210, an input interface module 220, an output interface module 230, and a communications module 240. As illustrated, the foregoing example modules of the example computing device 105 are in communication over a bus 250.

The processor 200 is a hardware processor. The processor 200 may, for example, be one or more ARM, Intel x86, PowerPC processors or the like.

The memory 210 allows data to be stored and retrieved. The memory 210 may include, for example, random access memory, read-only memory, and persistent storage. Persistent storage may be, for example, flash memory, a solid-state drive or the like. Read-only memory and persistent storage are a computer-readable medium. A computer-readable medium may be organized using a file system such as may be administered by an operating system governing overall operation of the example computing device 105.

The input interface module 220 allows the example computing device 105 to receive input signals. Input signals may, for example, correspond to input received from a user. The input interface module 220 may serve to interconnect the example computing device 105 with one or more input devices. Input signals may be received from input devices by the input interface module 220. Input devices may, for example, include one or more of a touchscreen input, keyboard, trackball or the like. In some embodiments, all or a portion of the input interface module 220 may be integrated with an input device. For example, the input interface module 220 may be integrated with one of the aforementioned exemplary input devices.

The output interface module 230 allows the example computing device 105 to provide output signals. Some output signals may, for example allow provision of output to a user. The output interface module 230 may serve to interconnect the example computing device 105 with one or more output devices. Output signals may be sent to output devices by an output interface module 230. Output devices may include, for example, a display screen such as, for example, a liquid crystal display (LCD), a touchscreen display. Additionally, or alternatively, output devices may include devices other than screens such as, for example, a speaker, indicator lamps (such as for, example, light-emitting diodes (LEDs)), and printers. In some embodiments, all or a portion of the output interface module 230 may be integrated with an output device. For example, the output interface module 230 may be integrated with one of the aforementioned example output devices.

The communications module 240 allows the example computing device 105 to communicate with other electronic devices and/or various communications networks. For example, the communications module 240 may allow the example computing device 105 to send or receive communications signals. Communications signals may be sent or received according to one or more protocols or according to one or more standards. For example, the communications module 240 may allow the example computing device 105 to communicate via a cellular data network, such as for example, according to one or more standards such as, for example, Global System for Mobile Communications (GSM), Code Division Multiple Access (CDMA), Evolution Data Optimized (EVDO), Long-term Evolution (LTE) or the like. The communications module 240 may allow the example computing device 105 to communicate using near-field communication (NFC), via Wi-Fi™, using Bluetooth™ or via some combination of one or more networks or protocols. In some embodiments, all or a portion of the communications module 240 may be integrated into a component of the example computing device 105. For example, the communications module may be integrated into a communications chipset.

Software comprising instructions is executed by the processor 200 from a computer-readable medium. For example, software may be loaded into random-access memory from persistent storage of memory 210. Additionally, or alternatively, instructions may be executed by the processor 200 directly from read-only memory of memory 210.

FIG. 3 depicts a simplified organization of software components stored in memory 210 of the example computing device 105. As illustrated these software components include an operating system 280 and an application 270.

The operating system 280 is software. The operating system 280 allows the application 270 to access the processor 200, the memory 210, the input interface module 220, the output interface module 230 and the communications module 240. The operating system 280 may be, for example, Apple's iOS™, Google's Android™, Linux™, Microsoft's Windows™, or the like.

The application 270 adapts the example computing device 105, in combination with the operating system 280, to operate as a device performing particular functions. For example, the application 270 may cooperate with the operating system 280 to adapt a suitable embodiment of the example computing device 105 to operate as the client device 110, the insurance server 140, the application evaluation server 170, and the application server(s) 180.

While a single application 270 is illustrated in FIG. 3, in operation, the memory 210 may include more than one application 270, and different applications 270 may perform different operations. For example, in embodiments where the computing device 105 is functioning as a client device 110, the application 270 may comprise a web browser application. The web browser application may be used, for example, to request to obtain various data on the client device 110. For example, a user may request, via a web browser application, to obtain an insurance rating for the user, query whether the user would qualify for insurance cover, or obtain an insurance premium for the user.

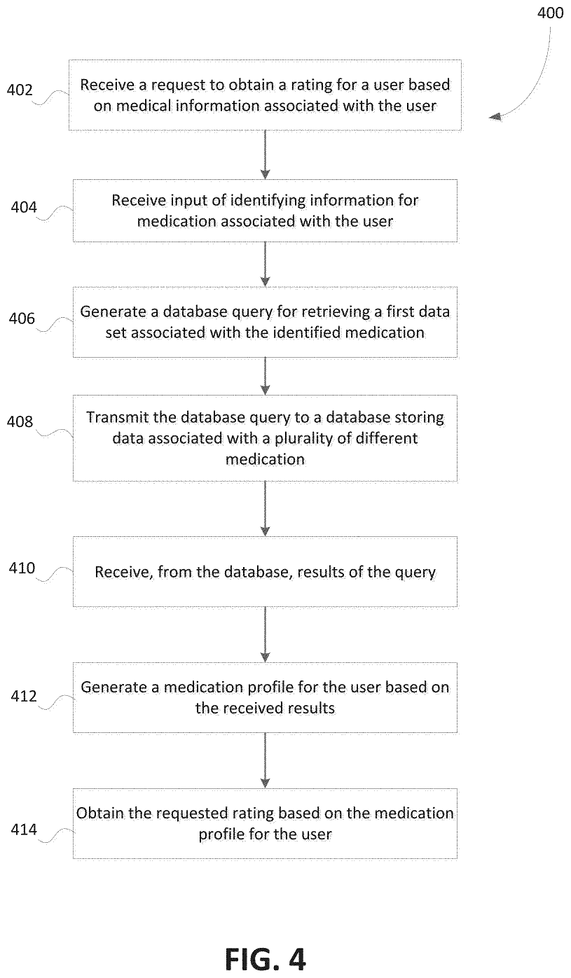

Reference is made to FIG. 4, which shows, in flowchart form, an example method 400 for processing database queries relating to medications for a user. Specifically, the method 400 allows for generating a medication profile for a user based on results of a query to obtain data associated with a user-inputted medication. The method 400 may be implemented, for example, in obtaining a rating that is requested using a client device associated with the user. For example, a computing system may obtain an insurance rating for a user whose medication profile is generated via the method 400.

Operations 402 and onward may be performed by one or more processors of a computing device such as, for example, the processor 200 (FIG. 2) of one or more suitably configured instances of the example computing device 105 (FIG. 2). The method 400 may be performed, for example, by a computing system, such as an insurance server, that is communicably connected to, at least, a client device and a medications database.

In operation 402, the computing system receives, from a client device, a request to obtain a rating for a user based on medical information associated with the user. The user may input the request via an application on the client device. For example, the user can request using a graphical user interface of an insurance application (or another application which can be used to obtain insurance policy data) for an insurance rating for the user. The insurance rating may be used, for example, by an insurance policy provider in deciding whether the user is eligible for insurance and/or to determine an insurance premium for the user.

The graphical user interface of the application may be provided by the computing system. In particular, the computing system may request, in operation 404, a user interface to be provided on the client device for inputting identifying information for prescribed medication associated with the user. For example, a user interface element for receiving text entry input may be provided on a graphical user interface of the application. The user may input text which uniquely identifies the user's medication, such as name of medication or drug identification number (DIN).

In some embodiments, the user interface may allow input of image data containing identifying information for the user's medication. That is, a user may capture images depicting information about the medication and provide the images as input to the computing system. As an example, the user may capture a photo of a label on a container or packaging for their medication and upload the photo via the user interface of an application on the client device.

The computing system then performs text recognition based on the received image data. For example, the image data or a portion thereof (such as a section or segment) may be analyzed to identify text contained therein. The image data may be processed by a parsing module of the computing system to extract one or more text entry items from the imaged object (e.g. medicine label). In some embodiments, when performing text recognition on the image data, the computing system may compare the image to one or more document templates from a templates database. The document templates may, for example, contain product labelling specifications for various products. The computing system may determine whether there is a match between the imaged object (e.g. label on a container or packaging) and one or more of the document templates from the templates database.

The received image data may first be passed to a field recognition engine, which determines regions and boundaries of the received image that correspond to the various data fields of an identified document type. The field recognition engine may, for example, perform a layout analysis by segmenting the image into regions having homogeneous content and assigning a logical meaning (e.g. association with a data field) to each of the regions. Additionally, or alternatively, the field recognition engine may employ a template matching technique to identify feature matches between the received image and document templates. Specifically, template matching can be used to identify regions of the received image that match data field labels and their neighboring regions in one or more document templates. By way of example, in some embodiments, the received image may be compared to one or more document templates, in order to identify matches of data fields. A data field in the received image may be identified by detecting a match with a data field in one of the templates based on, for example, dimensions/shape of the data field, text or graphics label associated with the data field, and/or relative location of the data field on the imaged document. Examples of data fields which are relevant in identifying medication include, among others, name of medication, drug identification number, dosages, expiry date, manufacture date, acceptable indications, uses, and medicinal ingredient identities.

Once the data field boundaries (and, accordingly, the corresponding data field regions) on the received image are identified, the image may be further processed by an optical character recognition (OCR) engine. The OCR engine is capable of converting images of typed, handwritten, or printed text into digital format, such as machine-encoded text. The OCR engine detects an image representation of a text entry item in a particular data field region and converts the image representation into text format. In this way, the text associated with the text entry items represented in the received image can be extracted.

In some embodiments, the OCR engine may be used in identifying data fields on the received image associated with the medication. In particular, the text content of a data entry item on the imaged object that is detected by the OCR engine may indicate or suggest the corresponding data field.

The text that is recognized from the image data may be deficient. In some embodiments, the computing system may be configured to determine that the recognized text does not contain text associated with at least one data field that is known to be included in a label for a medication. In response to determining that there is a deficiency in the recognized text, the computing system may generate display data for prompting a user of the client device to provide information relating to the missing one or more data fields. For example, the display data may be a graphical user interface including a fillable input form containing the at least one data field. Alternatively, the display data may be a graphical user interface including an application form having the missing data fields highlighted. The computing system may then transmit the display data to the client device in order to solicit additional information from the user(s) associated with the client device.

Once the identifying information for the user's medication is obtained, the computing system generates a database query for retrieving a first data set associated with the identified medication, in operation 406. The database query is for a data set containing one or more data parameters that are determined based on a type of the requested rating. Specifically, the data query may request for parameters that are relevant for the rating that is requested to be obtained by the user. For example, if an insurance rating for a user is desired to be obtained, the database query may request for information corresponding to, at least, a “medical condition” parameter, which identifies possible medical conditions associated with the identified medication, or a “mortality rate” parameter representing a rate of deaths among users of the identified medication.

In operation 408, the database query is transmitted to a database which stores data associated with a plurality of different medications. The computing system subsequently receives, from the database, results of the database query, in operation 410. The results include multiple possible values for at least one of the data parameters requested in the database query. For example, multiple possible health conditions may be obtained in the query results. Typically, a medication may be used to treat multiple different health conditions, and different dosages may be recommended for different health conditions. The results of the database query may identify a plurality of possible health conditions (e.g. illness, chronic condition, etc.) for the user.

In operation 412, the computing system generates a medication profile for the user based on the received results of the database query. The medication profile defines probabilities associated with the possible values for the data parameters of the results. That is, for one or more of the different possible values for parameters in the query results, probabilities may be independently assigned to the possible values. The probabilities may represent the likelihood that the medication is associated with the possible values. For example, the medication profile may define probabilities associated with the different possible health conditions associated with the user's medication. In particular, the medication profile may comprise a data structure containing probabilities corresponding to the different possible values identified in the query results.

In operation 414, the computing system obtains the requested rating based on the medication profile for the user. For example, an insurance rating for a user that is requested by the user's client device may be obtained based on the medication profile of the user. The computing system may itself obtain the insurance rating, or it may transmit, to a remote insurance server (such as insurance server 180), a request to obtain the insurance rating. The user may be algorithmically evaluated in order to obtain the requested insurance rating. The computing system may determine, based on the insurance rating, whether the user is eligible for insurance coverage. If the user is eligible, the computing system may also be configured to determine an insurance premium for the user.

In at least some embodiments, the insurance rating may be obtained without acquiring health conditions data associated with the user's medication. In particular, an insurance rating may be obtained based on mortality rate data for the user's medication, without first determining health conditions that are associated (e.g. treated) with the user's medication. Often, even though a medication may be used for more than one health condition, the severity of the conditions (which may be represented using mortality rates) associated with a given medication may be similar. Thus, the mortality rate data may be sufficient for obtaining an insurance rating. By obtaining insurance ratings based on mortality rates without retrieving associated health conditions data, there may be processing efficiencies for the computing system which allow for obtaining real-time rating data. For example, less processing resources may be consumed by the computing system when obtaining the insurance rating.

In those cases where the mortality rates for various health conditions associated with the medication vary significantly, the health conditions themselves may be considered in obtaining the insurance rating. In particular, if the mortality rates data is determined to be not sufficient for obtaining a reliable insurance rating, health conditions data may be retrieved and used in conjunction with the morality rates data in obtaining an insurance rating. For example, the computing system may prompt the user to provide selection of one or more of the health conditions which are associated with the user's medication, and use the selected health condition in obtaining an insurance rating.

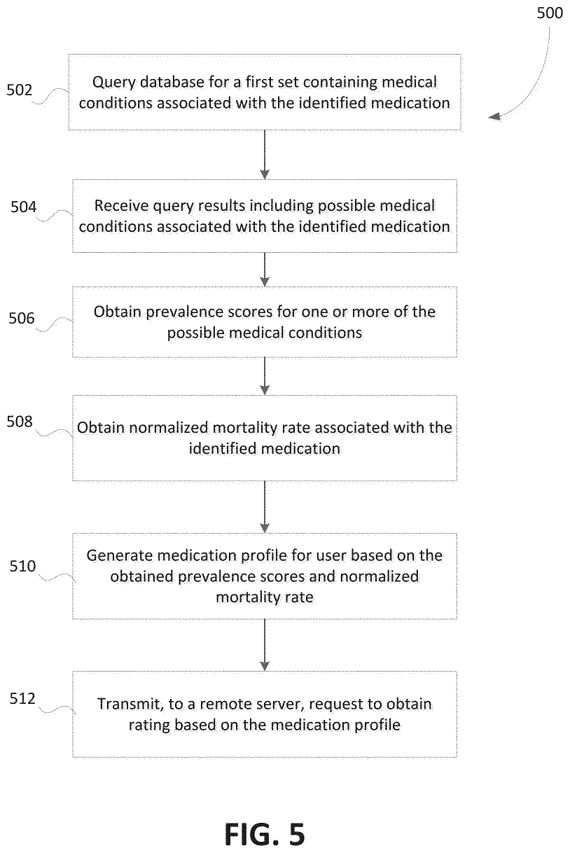

Reference is made to FIG. 5, which shows, in flowchart form, another example method 500 for processing database queries relating to medications for a user. Specifically, the method 500 allows for generating a medication profile for a user based on results of a query to obtain data associated with a user-inputted medication. The method 500 may be implemented, for example, in obtaining a rating that is requested using a client device associated with the user. For example, a computing system may obtain an insurance rating for a user whose medication profile is generated via the method 500.

Operations 502 and onward may be performed by one or more processors of a computing device such as, for example, the processor 200 (FIG. 2) of one or more suitably configured instances of the example computing device 105 (FIG. 2). The method 500 may be performed, for example, by a server system that is communicably connected to a client device, an insurance server, and a medications database.

In operation 502, the computing system queries a database for a first data set containing medical conditions associated with a user-identified medication. The database may, for example, be a medications database which contains identifiers of medications and medical information associated with the medication. The computing system receives results of the database query, which includes possible medical conditions that are associated with the identified medication, in operation 504.

In some embodiments, the prevalence of a medical condition, or how common the medical condition is, may be considered in attempting to identify a correct medical condition for the user. In operation 506, the computing system obtains prevalence scores for one or more of the possible medical conditions that are indicated in the query results, where the prevalence scores are numerical values representing the prevalence of the medical conditions. The prevalence scores may be predetermined and stored in a database, such as a medications database, and retrieved by the computing system.

In some embodiments, a single mortality rate may be determined for a user's medication. The single mortality rate may, for example, be a normalized mortality rate that is based on a plurality of mortality rates associated with the user's medication. For example, the normalized mortality rate may be a weighted average of the mortality rates identified for the different possible health conditions which may be treated using the user's medication. The weighting can be based on, for example, probabilities of the associated health conditions. Such probability data may be derived based on prevalence of the health conditions in a particular population. In operation 508, the computing system obtains a normalized mortality rate for the user's medication.

Upon obtaining the prevalence scores for the possible medical conditions and the normalized mortality rate, the computing system may generate a medication profile for the user. The medication profile may indicate the plurality of prevalence scores and the normalized mortality rate associated with the user's medication. Such medication profile may facilitate further processing of data downstream in obtaining a user rating based on their medications data. The computing system then transmits, to a remote server, a request to obtain a rating based on the medication profile generated in operation 510.

Other techniques for identifying the correct medical condition for a user based on the user's medication may be implemented by the computing system. For example, the computing system may present, in a user interface on the client device, options for inputting a selection of a medical condition. The computing system may transmit, to the client device, a request to provide such user interface on the client device. The user interface may display a list of possible medication conditions associated with an identified medication and prompt the user to select one of the listed medical conditions.

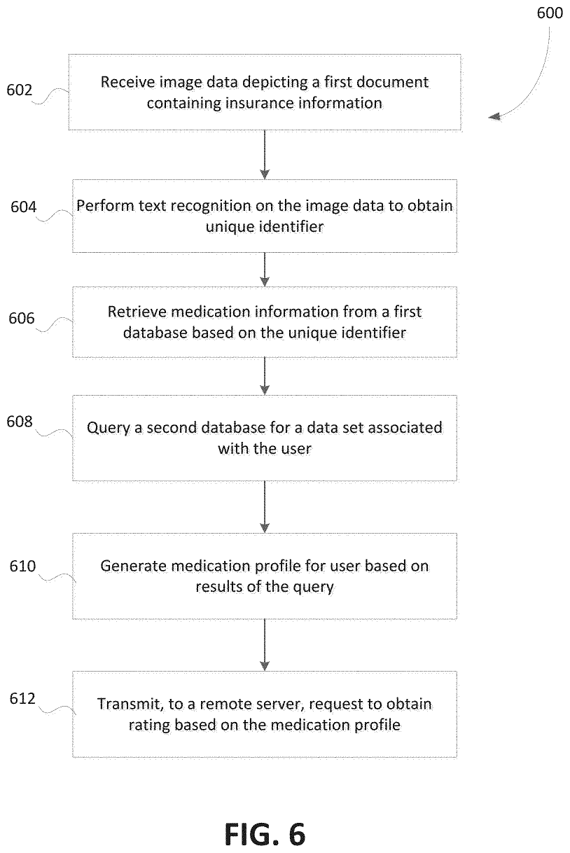

Reference is made to FIG. 6, which shows, in flowchart form, another example method 600 for processing database queries relating to medications for a user. Specifically, the method 600 allows for obtaining medication information based on image data depicting the user's medication and generating a medication profile for a user based on results of a query for medical information associated with the identified medication.

Operations 602 and onward may be performed by one or more processors of a computing device such as, for example, the processor 200 (FIG. 2) of one or more suitably configured instances of the example computing device 105 (FIG. 2). The method 600 may be performed, for example, by a server system that is communicably connected to a client device, an insurance server, and a medications database.

In operation 602, the computing system receives image data depicting a first document containing insurance information. The first document may, for example, be an insurance card of the user. The image of the first document may be uploaded through a user interface associated with the user's client device. The computing system may process the image data to extract identifying information for the user. For example, the computing system may perform text recognition on the image data to obtain a unique identifier for the user or the user's insurance account.

The computing system then retrieves medication information from a database based on the unique identifier, in operation 606. In particular, the computing system queries a second database for a data set associated with the user. For example, a third-party server associated with a health insurance program, such as a Medicare server, may store medications that are taken by a user. The computing system may query such server or database to retrieve the user's medications. For example, the computing system may make an application programming interface (API) call, by providing identifying information such as the unique identifier or the user's name.

The computing system may then generate a medications profile for the user based on the results of the database query, in operation 610. The medications profile may, for example, include a plurality of medications taken by the user and associated parameter values, such as medical conditions, mortality rates, etc., that are associated with each of one or more of the user's medications. The generated medication profile may then be transmitted to a remote insurance server in a request to obtain a rating for the user. That is, a request to obtain an insurance rating based on the medication profile is sent to an insurance server.

The various embodiments presented above are merely examples and are in no way meant to limit the scope of this application. Variations of the innovations described herein will be apparent to persons of ordinary skill in the art, such variations being within the intended scope of the present application. In particular, features from one or more of the above-described example embodiments may be selected to create alternative example embodiments including a sub-combination of features which may not be explicitly described above. In addition, features from one or more of the above-described example embodiments may be selected and combined to create alternative example embodiments including a combination of features which may not be explicitly described above. Features suitable for such combinations and sub-combinations would be readily apparent to persons skilled in the art upon review of the present application as a whole. The subject matter described herein and in the recited claims intends to cover and embrace all suitable changes in technology.

1. A computing system, comprising:
a communications module communicable with an external network;
a memory; and
a processor coupled to the communications module and the memory, the processor being configured to:
receive, from a client device, a request to obtain a rating for a user based on medical information associated with the user;
receive, via a user interface on the client device, input of identifying information for medication associated with the user;
generate a database query for retrieving a first data set associated with the identified medication, the first data set containing one or more data parameters that are determined based on a type of the requested rating;
transmit the database query to a database storing data associated with a plurality of different medications;
receive, from the database, results of the database query, the results including multiple possible values for at least one of the data parameters;
generate a medication profile for the user based on the received results, the medication profile defining probabilities associated with possible values for the at least one data parameter of the results;
obtain the requested rating based on the medication profile for the user.

2. The computing system of claim 1, wherein the one or more data parameters include a medical condition parameter identifying possible medical conditions associated with the identified medication.

3. The computing system of claim 2, wherein the processor is further configured to, after retrieving possible values for the medical condition parameter:
provide, to the client device, the possible medical conditions associated with the identified medication; and
receive, from the client device, input of user selection of a first medical condition from among the possible medical conditions.

4. The computing system of claim 3, wherein the medication profile includes, for each of at least one of the possible medical conditions, a prevalence score indicating a prevalence of the medical condition.

5. The computing system of claim 1, wherein the one or more data parameters include a mortality rate parameter identifying possible mortality rates associated with the identified medication.

6. The computing system of claim 1, wherein obtaining the requested rating comprises determining that the user is eligible for insurance coverage.

7. The computing system of claim 6, wherein obtaining the requested rating comprises determining an insurance premium for the user.

8. The computing system of claim 1, wherein the processor is further configured to transmit, to a server, a request to obtain medication information for the user.

9. The computing system of claim 1, wherein the user interface allows input of image data containing identifying information for the medication associated with the user.

10. The computing system of claim 9, wherein the processor is further configured to perform optical character recognition based on user inputted image data to obtain a unique identifier associated with the medication.

11. A processor-implemented method, comprising:
receiving, from a client device, a request to obtain a rating for a user based on medical information associated with the user;
receiving, via a user interface on the client device, input of identifying information for medication associated with the user;
generating a database query for retrieving a first data set associated with the identified medication, the first data set containing one or more data parameters that are determined based on a type of the requested rating;
transmitting the database query to a database storing data associated with a plurality of different medications;
receiving, from the database, results of the database query, the results including multiple possible values for at least one of the data parameters;
generating a medication profile for the user based on the received results, the medication profile defining probabilities associated with possible values for the at least one data parameter of the results;
obtaining the requested rating based on the medication profile for the user.

12. The method of claim 11, wherein the one or more data parameters include a medical condition parameter identifying possible medical conditions associated with the identified medication.

13. The method of claim 12, further comprising, after retrieving possible values for the medical condition parameter:
providing, to the client device, the possible medical conditions associated with the identified medication; and
receiving, from the client device, input of user selection of a first medical condition from among the possible medical conditions.

14. The method of claim 13, wherein the medication profile includes, for each of at least one of the possible medical conditions, a prevalence score indicating a prevalence of the medical condition.

15. The method of claim 11, wherein the one or more data parameters include a mortality rate parameter identifying possible mortality rates associated with the identified medication.

16. The method of claim 11, wherein obtaining the requested rating comprises determining that the user is eligible for insurance coverage.

17. The method of claim 16, wherein obtaining the requested rating comprises determining an insurance premium for the user.

18. The method of claim 11, further comprising transmitting, to a server, a request to obtain medication information for the user.

19. The method of claim 11, wherein the user interface allows input of image data containing identifying information for the medication associated with the user.

20. The method of claim 19, further comprising performing optical character recognition based on user inputted image data to obtain a unique identifier associated with the medication.
